(19) United States
(12) Patent Application Publication
(10) Pub. No.: US 2022/0082551 A1
(43) Pub. Date: Mar. 17, 2022

(54) SAMPLE ANALYZING METHOD AND SAMPLE ANALYZER

(71) Applicant: SHENZHEN MINDRAY BIO-MEDICAL ELECTRONICS CO., LTD., Shenzhen (CN)

(72) Inventors: Yong DAI, Shenzhen (CN); Qiushi YI, Shenzhen (CN); Zhonggang LIU, Shenzhen (CN)

(21) Appl. No.: 17/537,265

(22) Filed: Nov. 29, 2021

Related U.S. Application Data
(63) Continuation of application No. PCT/CN2019/090770, filed on Jun. 11, 2019.

(51) Int. Cl. G01N 33/49; G01N 15/14; G01N 21/47

(52) U.S. Cl. CPC G01N 33/4915; G01N 2015/1486; G01N 21/47; G01N 15/1459

(57) ABSTRACT

Disclosed are a sample analyzing method and a sample analyzer for measuring red blood cells and platelets. The method includes: preparing a first test sample solution containing a blood sample and a diluent; using an impedance method to acquire a first measurement result of red blood cells and platelets; when the first measurement result indicates that the blood sample is abnormal, preparing a second test sample solution containing the blood sample and a diluent or preparing a second test sample solution from the first test sample solution; irradiating the second test sample solution with light; collecting scattered light signals generated by particles in the second test sample solution; and acquiring a second measurement result of red blood cells and platelets in the second test sample solution based on the scattered light signals. Thus, RBC and PLT can be accurately classified especially under a condition of using an ordinary diluent.

### CROSS-REFERENCE TO RELATED APPLICATIONS

This application is a continuation of International Application No. PCT/CN2019/090770, filed Jun. 11, 2019, the content of which is incorporated herein by reference.

### TECHNICAL FIELD

The present application relates to the field of blood cell analysis, and specifically relates to a sample analyzing method for measuring red blood cells and platelets in a blood sample and a corresponding sample analyzer.

### BACKGROUND

Blood cell analysis is one of the analysis tests that are widely applied in hospital clinical testing. Parameters measured by the blood cell analysis can be divided into three types: leukon, erythron (red blood cells (RBC) and hemoglobin), and platelets (PLT). At present, an impedance method or an optical method is usually used for the measurement of RBCs and PLTs in the blood cell analysis.

However, both of the above two methods have some drawbacks. For example, in the impedance method, for some abnormal samples, such as samples having low PLT counts, there is often no clear boundary between a PLT histogram and an RBC histogram. As a result, the PLT histogram and the RBC histogram cannot be accurately separated by algorithm, and then cannot obtain an accurate PLT measurement result. Meanwhile, the measurement accuracy of samples having low PLT counts is a clinically important blood routine test index, and inaccurate measurement of samples having low PLT counts has become a main disadvantage of the impedance method. In addition, in a fluorescence method, a fluorescent dye is required to stain cells, and a special diluent is also required to spheronize cells, which result in an increased cost, and is not conducive to the promotion of the optical method in clinical practice.

Therefore, the current RBC and PLT analyzing methods have the above-mentioned problems, and need to be further improved.

### SUMMARY

A first aspect of the present application provides a sample analyzing method for measuring red blood cells and platelets in a blood sample, comprising:

preparing a first test sample solution containing a blood sample to be tested and a diluent;

flowing the first test sample solution in a flow cell having an aperture with electrodes, and detecting electrical signals generated when particles in the first test sample solution pass through the aperture;

obtaining, according to the electrical signals, a first measurement result of red blood cells and platelets in the first test sample solution;

determining, according to the first measurement result, whether the red blood cells and/or the platelets in the blood sample to be tested are abnormal;

when the first measurement result indicates that the red blood cells and/or the platelets in the blood sample to be tested are abnormal,
preparing a second test sample solution containing the blood sample to be tested and a diluent or preparing a second test sample solution from the first test sample solution;
irradiating the second test sample solution with light in an optical detection area;
collecting at least two types of scattered light signals generated by particles in the second test sample solution under light irradiation; and
obtaining, according to the at least two types of scattered light signals, a second measurement result of red blood cells and platelets in the second test sample solution.

In some embodiments, the first measurement result and/or the second measurement result may comprise a result of at least one parameter selected from a red blood cell count, a platelet count, a mean corpuscular volume, a mean platelet volume, and a red blood cell distribution width, or a result of a parameter calculated from a combination thereof.

In some embodiments, the abnormality may be that a number of the platelets in the first test sample solution is less than a predetermined threshold.

In some embodiments, the diluent is capable of maintaining morphology of the red blood cells and the platelets in the blood sample to be tested.

In some embodiments, the at least two types of scattered light signals may comprise at least two types of an axial light loss, forward-scattered light signals, medium-angle-scattered light signals, high-angle-scattered light signals, side-scattered light signals, and backward-scattered light signals.

In some embodiments, scattering angles of the axial light loss, the forward-scattered light signals, the medium-angle-scattered light signals, the high-angle-scattered light signals, the side-scattered light signals, and the backward-scattered light signals are 0° to 1°, 1° to 10°, 10° to 20°, 20° to 70°, 70° to 110°, and 110° to 160°, respectively.

In some embodiments, the at least two types of scattered light signals may comprise at least one type, particularly at least two types, of the forward-scattered signals, the medium-angle-scattered signals, and the high-angle-scattered signals. Preferably, the at least two types of scattered light signals may comprise the forward-scattered signals and the medium-angle-scattered signals, or the forward-scattered signals and the high-angle-scattered signals.

In some embodiments, the light irradiation may be polarized light irradiation, and the at least two types of scattered light signals comprise at least two types of signals in specific states of polarization of axial light loss, forward-scattered light signals, medium-angle-scattered light signals, high-angle-scattered light signals, side-scattered light signals, and backward-scattered light signals that are generated by the particles in the second test sample solution under the polarized light irradiation. In some embodiments, the at least two types of scattered light signals may comprise at least one type, particularly at least two types, of the signals in specific states of polarization of the forward-scattered signals, the medium-angle-scattered signals, and the high-angle-scattered signals.

In some embodiments, the step of obtaining, according to the at least two types of scattered light signals, a second measurement result of red blood cells and platelets in the second test sample solution may comprise:

generating, according to the at least two types of scattered light signals, a two-dimensional or three-dimensional scattergram of the particles in the second test sample solution; and

obtaining, based on the two-dimensional or three-dimensional scattergram, the second measurement result of the red blood cells and the platelets in the second test sample solution.

In some embodiments, the method may further comprise: when the first measurement result indicates that the red blood cells and/or the platelets in the blood sample to be tested are abnormal, obtaining, according to the first measurement result and the second measurement result, a final measurement result of the red blood cells and the platelets in the blood sample to be tested, for example, a final measurement result of at least one parameter selected from a red blood cell count, a platelet count, a mean corpuscular volume, a mean platelet volume, and a red blood cell distribution width, or a final measurement result of a parameter calculated from a combination thereof; or determining the second measurement result as a final measurement result of the red blood cells and the platelets in the blood sample to be tested.

In some embodiments, the method may further comprise: when the first measurement result indicates that the red blood cells and/or the platelets in the blood sample to be tested are normal, determining the first measurement result as a final measurement result of the red blood cells and the platelets in the blood sample to be tested.

In some embodiments, the method may further comprise: outputting the final measurement result of the red blood cells and the platelets of the blood sample to be tested.

A second aspect of the present application provides a sample analyzer, comprising:

a sampling apparatus having a pipette with a pipette nozzle and having a driving apparatus for driving the pipette to quantitatively aspirate a blood sample through the pipette nozzle;

a sample preparation apparatus having a reaction cell and a liquid supply part, wherein the reaction cell is configured to receive the blood sample aspirated by the sampling apparatus, and the liquid supply part supplies a diluent to the reaction cell, such that the blood sample aspirated by the sampling apparatus is mixed in the reaction cell with the diluent supplied by the liquid supply part, to prepare a test sample solution;

an impedance detection apparatus comprising a first flow cell having an aperture with electrodes, wherein the impedance detection apparatus is configured to detect DC impedances generated when particles in the test sample solution pass through the aperture, and output electrical signals reflecting information about the passage of the particles through the aperture;

an optical detection apparatus having a light source, a second flow cell, and optical collectors, wherein the particles in the test sample solution that has been treated with the diluent are capable of flowing in the flow cell, light emitted by the light source irradiates the particles in the second flow cell to generate at least two types of scattered light signals, and the optical collectors are configured to collect the at least two types of scattered light signals;

a transfer apparatus configured to transfer the test sample solution that has been treated with the diluent in the reaction cell to the impedance detection apparatus and the optical detection apparatus; and

a processor communicatively connected to the sampling apparatus, the sample preparation apparatus, the impedance detection apparatus, the optical detection apparatus, and the transfer apparatus and configured to:
instruct the sample preparation apparatus to prepare a first test sample solution containing a blood sample to be tested and a diluent;
instruct the transfer apparatus to transfer the prepared first test sample solution to the first flow cell;
obtain, from the impedance detection apparatus, electrical signals generated when the first test sample solution passes through the first flow cell of the impedance detection apparatus;
obtain, according to the electrical signals, a first measurement result of red blood cells and platelets in the first test sample solution;
determine, according to the first measurement result, whether the red blood cells and/or the platelets in the blood sample to be tested are abnormal;
when the first measurement result indicates that the red blood cells and/or the platelets in the blood sample to be tested are abnormal,
   instruct the sample preparation apparatus to prepare a second test sample solution containing the blood sample to be tested and a diluent or prepare a second test sample solution from the first test sample solution;
   instruct the transfer apparatus to transfer the prepared second test sample solution to the second flow cell;
   obtain, from the optical detection apparatus, at least two types of scattered light signals generated by particles in the second test sample solution in the second flow cell of the optical detection apparatus under light irradiation, and
   obtain, according to the at least two types of scattered light signals, a second measurement result of red blood cells and platelets in the second test sample solution.

In some embodiments, the first measurement result and/or the second measurement result may comprise a final measurement result of at least one parameter selected from a red blood cell count, a platelet count, a mean corpuscular volume, a mean platelet volume, and a red blood cell distribution width, or a final measurement result of a parameter calculated from a combination thereof.

In some embodiments, the abnormality may be that a number of the platelets in the first test sample solution is less than a predetermined threshold.

In some embodiments, the first flow cell and the second flow cell may be configured as the same flow cell having an aperture with electrodes.

In some embodiments, the diluent is capable of maintaining morphology of the red blood cells and the platelets in the blood sample.

In some embodiments, the at least two types of scattered light signals may comprise at least two types of axial light loss, forward-scattered light signals, medium-angle-scattered light signals, high-angle-scattered light signals, side-scattered light signals, and backward-scattered light signals.

In some embodiments, scattering angles of the axial light loss, the forward-scattered light signals, the medium-angle-scattered light signals, the high-angle-scattered light signals, the side-scattered light signals, and the backward-scattered light signals are 0° to 1°, 1° to 10°, 10° to 20°, 20° to 70°, 70° to 110°, and 110° to 160°, respectively.

In some embodiments, the at least two types of scattered light signals may comprise at least one type, particularly at least two types, of the forward-scattered signals, the medium-angle-scattered signals, and the high-angle-scattered signals. Preferably, the at least two types of scattered light signals may comprise the forward-scattered signals and the medium-angle-scattered signals, or the forward-scattered signals and the high-angle-scattered signals.

In some embodiments, the light source may be configured as a light source emitting polarized light, and the at least two types of scattered light signals comprise at least two types of signals in specific states of polarization of axial light loss, forward-scattered light signals, medium-angle-scattered light signals, high-angle-scattered light signals, side-scattered light signals, and backward-scattered light signals that are generated by the particles in the second test sample solution under the polarized light irradiation.

In some embodiments, the at least two types of scattered light signals comprise at least one type, particularly at least two types, of the signals in specific states of polarization of the forward-scattered signals, the medium-angle-scattered signals, and the high-angle-scattered signals.

In some embodiments, the processor may be further configured to:

generate, according to the at least two types of scattered light signals, a two-dimensional or three-dimensional scattergram of the particles in the second test sample solution; and

obtain, based on the two-dimensional or three-dimensional scattergram, the second measurement result of the red blood cells and the platelets in the second blood sample to be tested.

In some embodiments, the processor may be further configured to: when the first measurement result indicates that the red blood cells and/or the platelets in the blood sample to be tested are abnormal, obtain, according to the first measurement result and the second measurement result, a final measurement result of the red blood cells and the platelets in the blood sample to be tested, for example, a final measurement result of at least one parameter selected from a red blood cell count, a platelet count, a mean corpuscular volume, a mean platelet volume, and a red blood cell distribution width, or a final measurement result of a parameter calculated from a combination thereof; or determine the second measurement result as a final measurement result of the red blood cells and the platelets in the blood sample to be tested.

In some embodiments, the processor may be further configured to: when the first measurement result indicates that the red blood cells and/or the platelets in the blood sample to be tested are normal, determine the first measurement result as a final measurement result of the red blood cells and the platelets in the blood sample to be tested.

In some embodiments, the sample analyzer may further comprise an output apparatus communicatively connected to the processor and configured to output the final measurement result of the red blood cells and the platelets of the blood sample to be tested.

The method for measuring red blood cells and platelets in a blood sample and the sample analyzer according to the embodiments of the present application can achieve accurate classification and/or counting of RBCs and PLTs, especially under a condition of using an ordinary diluent. In particular, the present application can improve the accuracy of measurement using the impedance method, and achieve the accurate classification and/or counting of RBCs and PLTs of an abnormal sample using a general diluent.

### BRIEF DESCRIPTION OF THE DRAWINGS

In order to more clearly describe the technical solutions in the embodiments of the present application or in the prior art, the drawings required for describing the embodiments or the prior art will be briefly described below. Apparently, the drawings in the following description show only some of the embodiments of the present application, and those of ordinary skill in the art may still derive other drawings from these drawings without creative efforts.

FIG. 1 is a histogram obtained by measuring RBCs and PLTs using an impedance method;

FIG. 2A is an RBC histogram obtained by measuring RBCs and PLTs in a normal sample using an impedance method;

FIG. 2B is a PLT histogram obtained by measuring RBCs and PLTs in a normal sample using an impedance method;

FIG. 3 is a PLT histogram obtained by measuring RBCs and PLTs in an abnormal sample using an impedance method;

FIG. 4 is a scattergram of forward-scattered light signals (FSC) versus fluorescence signals (FL) obtained by measuring RBCs and PLTs in a blood sample using a fluorescence method;

FIG. 5 is a schematic structural diagram of a sample analyzer having an optical detection apparatus according to an embodiment of the present application;

FIG. 6 is a schematic structural diagram of a sample analyzer having an impedance detection apparatus according to another embodiment of the present application;

FIG. 7 is a schematic flowchart of a sample analyzing method for measuring red blood cells and platelets in a blood sample according to a first implementation of the present application;

FIG. 8 is a schematic diagram of scattering angles of various types of scattered light signals according to an embodiment of the present application;

FIG. 9 is a scattergram of forward-scattered light signals (FSC) versus medium-angle-scattered light signals (MAS) obtained according to the sample analyzing method according to the first implementation of the present application;

FIG. 10 is a scattergram of forward-scattered light signals (FSC) versus high-angle-scattered light signals (WAS) obtained according to the sample analyzing method according to the first implementation of the present application; and

FIG. 11 is a schematic flowchart of a sample analyzing method for measuring red blood cells and platelets in a blood sample according to a second implementation of the present application.

### DETAILED DESCRIPTION

In order to make the objectives, technical solutions, and advantages of the present application more clear, example embodiments according to the present application will be described in detail below with reference to the accompanying drawings. Apparently, the described embodiments are merely some rather than all of the embodiments of the present application. It should be understood that the example embodiments described herein do not constitute any limitation to the present application. All other embodiments derived by those skilled in the art without creative efforts on the basis of the embodiments of the present application described in the present application shall fall within the scope of protection of the present application.

In the following description, a large number of specific details are given to provide a more thorough understanding of the present application. However, it would be understood by those skilled in the art that the present application can be implemented without one or more of these details. In other examples, to avoid confusion with the present application, some technical features known in the art are not described.

It should be understood that the present application can be implemented in different forms and should not be construed as being limited to the embodiments presented herein. On the contrary, these embodiments are provided to make the disclosure thorough and complete, and to fully convey the scope of the present application to those skilled in the art.

The terms used herein are intended only to describe specific embodiments and do not constitute a limitation to the present application. When used herein, the singular forms of “a”, “an”, and “said/the” are also intended to include plural forms, unless the context clearly indicates otherwise. It should also be appreciated that the terms “comprise” and/or “include”, when used in the specification, determine the existence of described features, integers, steps, operations, elements, and/or components, but do not exclude the existence or addition of one or more other features, integers, steps, operations, elements, components, and/or groups thereof. As used herein, the term “and/or” includes any and all combinations of relevant listed items.

For a thorough understanding of the present application, detailed steps and detailed structures will be provided in the following description to explain the technical solutions proposed by the present application. The preferred embodiments of the present application are described in detail as follows. However, in addition to these detailed descriptions, the present application may further have other implementations.

At present, an impedance method or a fluorescence optical method is usually used in a hematology analyzer to measure RBCs and PLTs in a blood sample.

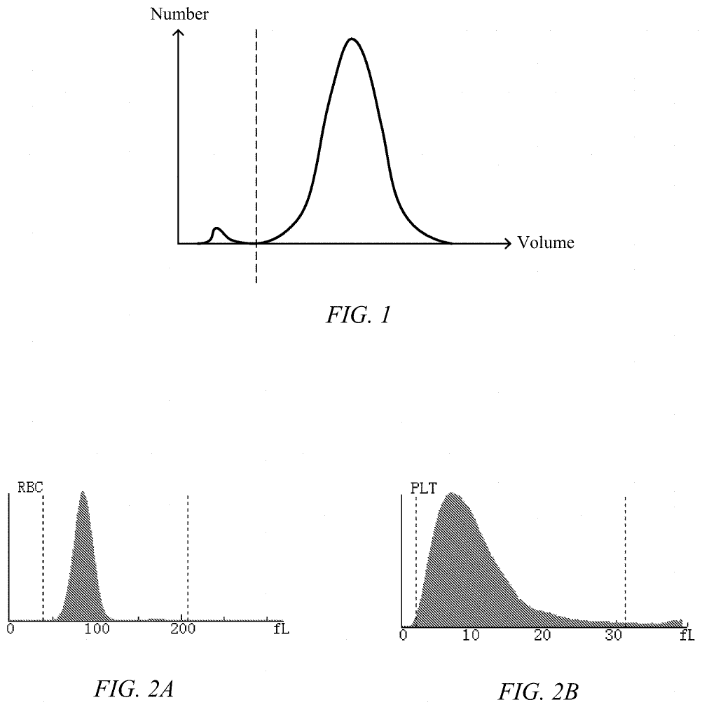

In the impedance method based on the Coulter principle, a diluted blood sample is passed through an aperture, cross which a constant current source is applied, and each cell passing through the aperture causes a change in electrical impedance of a liquid in the aperture, thereby generating an electrical impulse. A corresponding electrical impulse is obtained, an amplitude of which represents a volume of a cell, so that an RBC and PLT histogram is generated. The histogram presents one-dimensional information, that is, only volume information of cells, as shown in FIG. 1.

In FIG. 1, a PLT histogram is on the left of the dashed line, and an RBC histogram is on the right of the dashed line. A diameter of an RBC particle is approximately 3 times a diameter of a PLT particle, and the number of RBC particles is approximately 30 times the number of PLT particles. Therefore, the PLT histogram is on the left of the RBC histogram, and an area enclosed by the PLT histogram and the horizontal axis is far smaller than that enclosed by the RBC histogram and the horizontal axis. In other words, the dashed line in FIG. 1 is a boundary between the RBC histogram and the PLT histogram. The RBC histogram and the PLT histogram are normalized and drawn separately, to form an RBC histogram and a PLT histogram that are common in a blood cell analyzer, as shown in FIGS. 2A and 2B. FIG. 2A is an RBC histogram obtained by measuring RBCs and PLTs of a normal sample using the impedance method; and FIG. 2B is a PLT histogram obtained by measuring RBCs and PLTs of a normal sample using the impedance method.

In RBC and PLT histograms of a normal sample, there is a clear boundary between an RBC peak and a PLT peak. Generally, a trough on the right of a main peak of PLT particles in the PLT histogram is used as a boundary between PLTs and RBCs, and then the two histograms are analyzed separately, to obtain related measurement parameters of PLTs and RBCs. However, for some abnormal samples, such as samples having low PLT count, there is often no clear boundary between a PLT histogram and an RBC histogram. As shown in FIG. 3, a low-value PLT histogram is jagged, and as a result, the PLT histogram and the RBC histogram cannot be accurately separated by an algorithm, and then an accurate PLT measurement result cannot be obtained. The accuracy of samples having low PLT counts is a clinically important blood routine test index, and inaccurate measurement of samples having low PLT counts is a main disadvantage of the measurement using the impedance method.

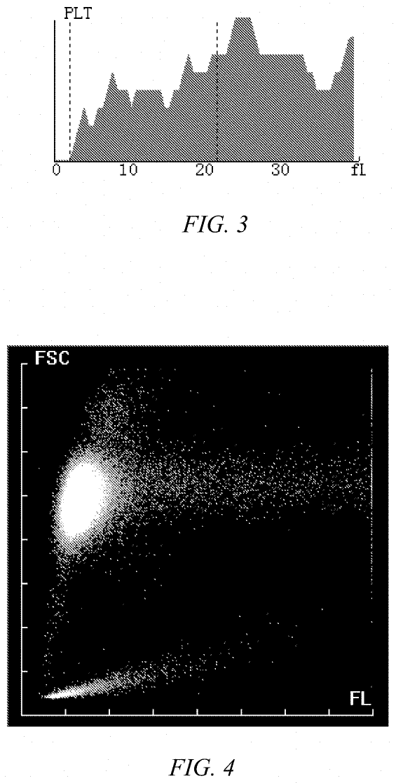

However, the fluorescence optical method can overcome this defect. The fluorescence method is based on flow cytometry. Diluted and stained samples are conveyed by a sheath flow through an optical detection area in sequence. After each cell is irradiated by an excitation light source, a forward-scattered signal (representing a volume of the cell) and a fluorescence signal (representing nucleic acid content in the cell) are obtained in an optical system, so as to generate a two-dimensional scattergram, for distinguishing and calculation of RBCs and the PLTs. Compared with the one-dimensional histogram obtained using the impedance method, the two-dimensional scattergram of the optical method has one more dimension of information, so that PLTs and the RBCs can be accurately distinguished on the two-dimensional scattergram, as shown in FIG. 4. In the fluorescence method, a fluorescent dye is required to stain cells, and a special diluent is also required to spheronize cells. Therefore, the cost is high, which is not conducive to the promotion of the optical method in clinical practice.

To solve this problem, the present application proposes a sample analyzing method and a sample analyzer that use only scattered light information to detect red blood cells and platelets in a blood sample. The sample analyzing method and the sample analyzer can achieve accurate classification and counting of RBCs and PLTs by using a general diluent, especially can achieve accurate classification and counting of RBCs and PLTs of an abnormal sample by using a general diluent. In addition, in application scenarios with the absence of a plurality of diluents (especially spherical diluents), for abnormal samples, the method and the sample analyzer proposed in the present application can further improve the accuracy of the measurement using the impedance method.

The sample analyzing method and the sample analyzer for measuring red blood cells and platelets in a blood sample that are provided in the present application will be described in detail below with reference to the accompanying drawings.

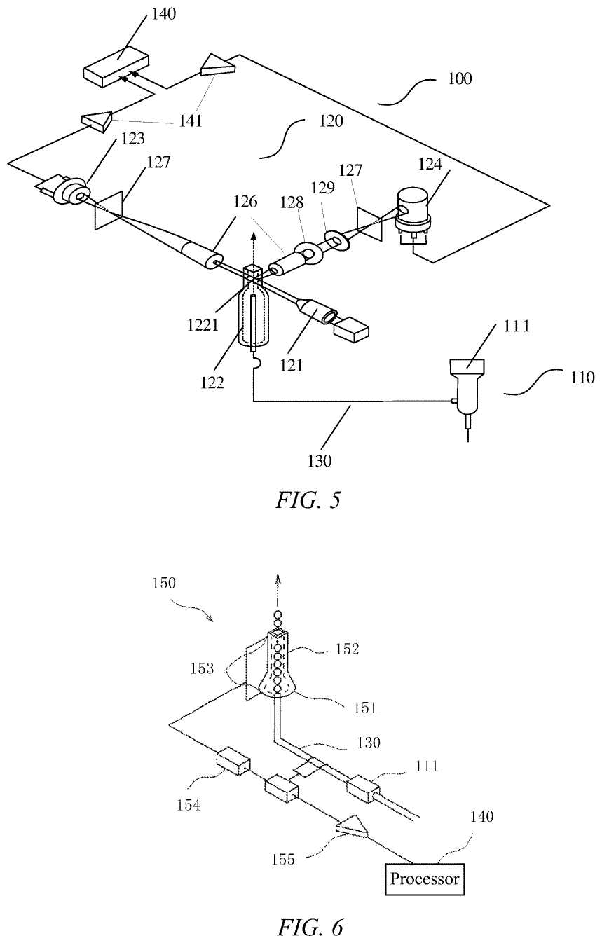

First, the sample analyzer provided in the present application will be described in detail with reference to FIGS. 5 and 6.

As shown in FIG. 5, the sample analyzer 100 comprises at least a sampling apparatus (not shown), a sample preparation apparatus 110, an optical detection apparatus 120, a transfer apparatus 130, and a processor 140.

The sampling apparatus has a pipette with a pipette nozzle and has a driving apparatus for driving the pipette to quantitatively aspirate a blood sample through the pipette nozzle. Further, after aspirating the blood sample, the sampling apparatus is driven and moved by its driving apparatus to a reaction cell 111 of the sample preparation apparatus 110, and injects the aspirated blood sample into the reaction cell 111.

The sample preparation apparatus 110 has at least one reaction cell 111 and has a liquid supply part (not shown), wherein the reaction cell 111 is configured to receive the blood sample aspirated by the sampling apparatus, and the liquid supply part supplies a diluent to the reaction cell 111, such that the blood sample aspirated by the sampling apparatus reacts with the diluent supplied by the liquid supply part in the reaction cell, to prepare a test sample solution. For example, the liquid supply part may be configured to inject an appropriate diluent into the reaction cell to process particles in the blood sample, thereby preparing the test sample solution, for subsequent measurement. The diluent may be a general diluent necessary for a blood cell analyzer to maintain normal morphology of red blood cells and platelets in blood sample, and a special diluent that spheronizes RBCs and PLTs is not required. For example, the diluent may comprise components such as sodium chloride, phosphate buffer solution, or preservatives. The diluent is not limited to one of them, but may be selected as required, and details are not described herein.

The optical detection apparatus 120 has a light source 121, a flow cell 122, and optical collectors 123 and 124. The light source 121 may emit natural light or light of a specific wavelength band, which is not limited to one of them. Alternatively, the light source 121 may be a polarized light source to emit polarized light in a specific state of polarization. The flow cell 122 has an orifice 1221, and particles in the test sample solution that has been treated with the diluent in the sample preparation apparatus 110 can flow in the flow cell 122 and pass through the orifice 1221 one by one. The light emitted by the light source 121 irradiates the particles in the flow cell 122 to generate optical signal information. The optical collectors 123 and 124 are configured to collect the optical signal information. The optical signal information may comprise at least two types of axial light loss, forward-scattered light signals, medium-angle-scattered light signals, high-angle-scattered light signals, side-scattered light signals, and backward-scattered light signals. When a polarized light is used for irradiation, the optical signal information comprises at least two types of signals in specific states of polarization of the axial light loss, the forward-scattered light signals, the medium-angle-scattered light signals, the high-angle-scattered light signals, the side-scattered light signals, and the backward-scattered light signals. In other words, the optical detection apparatus 120 comprises at least two optical collectors selected from an axial light loss optical collector, a forward-scattered light signal optical collector, a medium-angle-scattered light signal optical collector, a high-angle-scattered light signal optical collector, a side-scattered light signal optical collector, and a backward-scattered light signal optical collector.

In an embodiment, the optical collectors are configured as photoelectric detectors, such as photodiodes or photomultipliers. Specifically, as shown in FIG. 5, forward-scattered light emitted by blood cells flowing in the flow cell 122 passes through a condenser 126 and a pinhole 127 and is then received by the photodiode (forward-scattered light optical collector) 123, while side-scattered light passes through the condenser 126, a dichroic mirror 128, an optical film 129, and the pinhole 127, and is then received by the photomultiplier (side-scattered light optical collector) 124. Optical signals output from the optical collectors 123 and 124 undergo amplification, waveform processing, and other analog signal processing performed by amplifiers 141 respectively, and then are transferred to the processor 140.

In the present application, scattering angles of the axial light loss, the forward-scattered light signals, the medium-angle-scattered light signals, the high-angle-scattered light signals, the side-scattered light signals, and the backward-scattered light signals are 0° to 1°, 1° to 10°, 10° to 20°, 20° to 70°, 70° to 110°, and 110° to 160°, respectively. The axial light loss optical collector, the forward-scattered light signal optical collector, the medium-angle-scattered light signal optical collector, the high-angle-scattered light signal optical collector, and the side-scattered light signal optical collector are configured to receive scattered light signals at the above-mentioned scattering angles, respectively.

Preferably, the optical detection apparatus 120 comprises at least two of the forward-scattered light signal optical collector, the medium-angle-scattered light signal optical collector, and the high-angle-scattered light signal optical collector, to receive at least two types of the forward-scattered signals, the medium-angle-scattered signals, and the high-angle-scattered signals, or at least two types of signals in specific states of polarization of the forward-scattered signals, the medium-angle-scattered signals, and the high-angle-scattered signals, so as to improve the accuracy of the measurement result of the red blood cells and the platelets.

The transfer apparatus 130 is configured to transfer the test sample solution that has been treated with the diluent in the reaction cell 111 to the optical detection apparatus 120.

The processor 140 is communicatively connected to the sampling apparatus, the sample preparation apparatus 110, the optical detection apparatus 120, and the transfer apparatus 130 and is configured to obtain the optical signal information from the optical detection apparatus 120 and process the optical signal information, so as to obtain a measurement result of the particles in the blood sample to be tested. The processor 140 may have an A/D converter (not shown) configured to convert an analog signal provided by the optical detection apparatus 120 into a digital signal. Specifically, the processor 140 is configured to implement the sample analyzing method according to the present application, which will be further described in detail below.

In some embodiments of the present application, the processor 140 may be at least one of an application-specific integrated circuit (ASIC), a digital signal processor (DSP), a digital signal processing device (DSPD), a programmable logic device (PLD), a field programmable gate array (FPGA), a central processing unit (CPU), a controller, a microcontroller, and a microprocessor. It can be understood that for different devices, other electronics may also implement functions of the processor described above and are not specifically limited in the embodiments of the present application.

In addition, as shown in FIG. 6, the sample analyzer 100 may further comprise an impedance detection apparatus 150, the impedance detection apparatus comprising a flow cell 151 having an aperture 152 with electrodes 153, wherein the impedance detection apparatus 150 detects DC impedances generated when particles in the test sample solution pass through the aperture 152, and outputs electrical signals reflecting information about the passage of the particles through the aperture.

Specifically, after aspirating the blood sample, the sampling apparatus is driven and moved by its driving apparatus to the reaction cell 111 of the sample preparation apparatus 110, and injects the aspirated blood sample into the reaction cell 111. The transfer apparatus 130 may further transfer the test sample solution that has been treated with the diluent in the reaction cell 111 to the impedance detection apparatus 150, that is, to the flow cell 151. The impedance detection apparatus 150 may be further provided with a sheath fluid chamber (not shown) configured to supply a sheath fluid to the flow cell 151. In the flow cell 152, the test sample solution is wrapped by the sheath fluid to flow, and the aperture 152 turns the flow of the test sample solution into a tiny stream, such that the particles (visible components) contained in the test sample solution pass through the aperture 152 one by one. The electrodes 153 are electrically connected to a DC power supply 154, and the DC power supply 154 supplies DC power between the electrodes 153. When the DC power supply 154 supplies DC power, impedance between the electrodes 153 can be detected. Resistance signals representing changes in impedance are amplified by the amplifier 155 and then transferred to the processor 140. The strength of the resistance signals corresponds to the volumes (sizes) of the particles. Therefore, the processor 140 performs signal processing on the resistance signals to obtain a classification and counting result of the particles in the test sample solution, especially a classification and counting results of red blood cells and platelets.

In an advantageous embodiment, the flow cell 122 of the optical detection apparatus 120 and the flow cell 152 of the impedance detection apparatus 150 may be the same flow cell to save space. In other words, the flow cell 152 of the impedance detection apparatus 150 may also be used as the flow cell 122 of the optical detection apparatus 120. Certainly, the flow cell 122 of the optical detection apparatus 120 and the flow cell 152 of the impedance detection apparatus 150 may alternatively be two separate flow cells.

In addition, the blood sample analyzer 100 may further comprise an output apparatus (not shown) communicatively connected to the processor 140, the output apparatus being configured to receive and display a blood sample analysis result and/or a scattergram generated by at least two types of optical signal information from the processor 140.

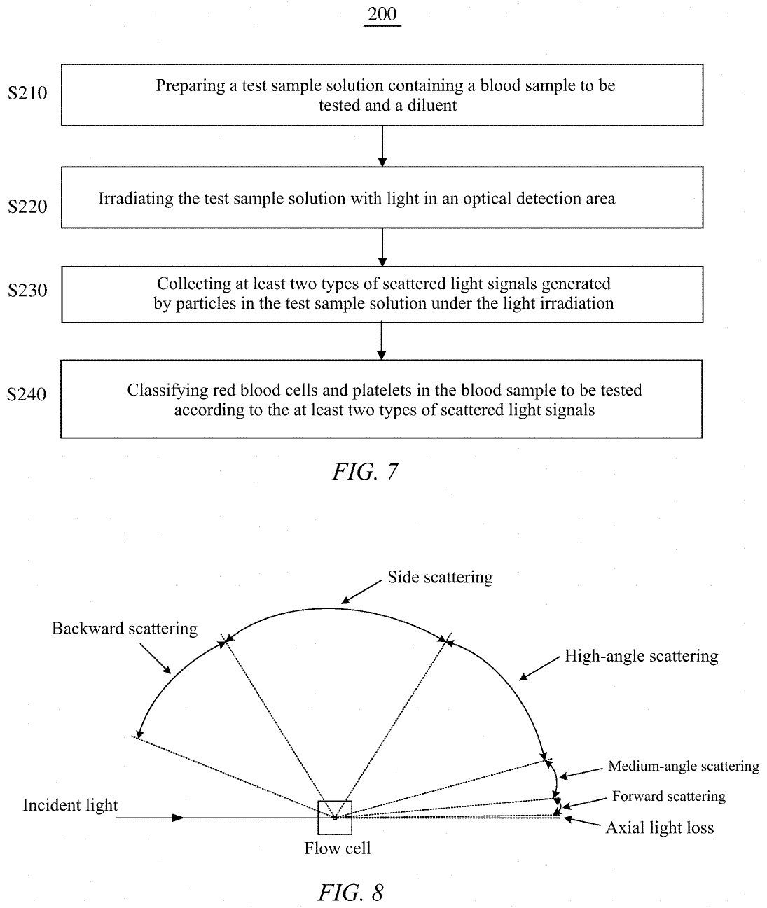

Next, a specific method and principle for measuring red blood cells and platelets in a blood sample by the foregoing blood sample analyzer 100 will be described in detail with reference to FIGS. 7 to 11.

FIG. 7 shows a sample analyzing method for measuring red blood cells and platelets in a blood sample according to a first implementation of the present application. As shown in FIG. 7, the sample analyzing method 200 for measuring red blood cells and platelets in a blood sample comprises the following steps:

Step S210: a test sample solution containing a blood sample to be tested and a diluent is prepared.

For example, in this step, the test sample solution is prepared, for example, in the sample preparation apparatus 110 of the sample analyzer 100. The sample preparation apparatus 110 has at least one reaction cell 111 and has a liquid supply part (not shown). Under the control of the processor 140, after aspirating the blood sample, the sampling apparatus is driven and moved by its driving apparatus to the reaction cell 111 of the sample preparation apparatus 110, and then the liquid supply part supplies the diluent to the reaction cell 111, such that the blood sample aspirated by the sampling apparatus reacts with the diluent supplied by the liquid supply part in the reaction cell, to prepare the test sample solution. The diluent provides appropriate pH, conductivity, and osmotic pressure for the blood sample to be tested, thereby ensuring the morphological integrity of cells without hemolysis. The diluent is further used to clean residual substances of a previously tested sample, thereby ensuring the cleanliness of sampling needle, pipeline, and flow cell of the sample analyzer and preventing cross-contamination. In addition, when the sample analyzer is temporarily stopped, the diluent should be filled in the pipeline to prevent foreign matters such as external dust from entering the instrument and causing a malfunction. The diluent may be a general diluent necessary for a blood cell analyzer to maintain normal morphology of red blood cells and platelets in a blood sample, instead of a special diluent that spheronizes RBCs and PLTs.

In an embodiment of the present application, the diluent may comprise components such as sodium chloride, phosphate buffer solution, or preservatives. The diluent is not limited to one of them, but may be selected as required, and details are not described herein.

Step S220: the test sample solution is irradiated with light in an optical detection area.

For example, in this step, under the control of the processor 140, the transfer apparatus 130 of the sample analyzer 100 transfers the test sample solution that has been treated with the diluent in the reaction cell 111 to the flow cell 122 of the optical detection apparatus 120. Particles in the test sample solution that has been treated with the diluent can flow in the flow cell 122 and pass through the orifice 1221 one by one. And the light emitted by the light source 121 irradiates the particles in the flow cell 122 to generate optical signal information.

Step S230: at least two types of scattered light signals, that is, scattered light signals at specific scattering angles, generated by the particles in the test sample solution under the light irradiation are collected.

For example, in this step, under the control of the processor 140, the optical detection apparatus 120 transfers the scattered light signals output by the optical detection apparatus to the processor 140, so that the processor 140 processes the scattered light signals. In this embodiment of the present application, the scattered light signals may comprise at least two types of axial light loss, forward-scattered light signals, medium-angle-scattered light signals, high-angle-scattered light signals, side-scattered light signals, and backward-scattered light signals. Preferably, the scattered light signals comprise at least one type, particularly at least two types, of the forward-scattered signals, the medium-angle-scattered signals, and the high-angle-scattered signals. More preferably, the at least two types of scattered light signals may comprise the forward-scattered signals and the medium-angle-scattered signals, or the forward-scattered signals and the high-angle-scattered signals.

The scattering angle in this embodiment of the present application is an angle defined by a vertex and two sides, wherein the vertex of the angle is the center of an overlapping area between a sample flow in the flow cell and an excitation light beam, a first side of the angle is a propagation direction of the excitation light beam, and a second side of the angle is a propagation direction of scattered light emitted by a particle at the above-mentioned vertex. Unless specified otherwise, reference is always made to this explanation for the scattering angle.

Herein, the present application defines the scattering angles of the scattered light signals as follows, as shown in FIG. 8: a scattering angle of the axial light loss is 0° to 1°; a scattering angle of the forward-scattered light signal is 1° to 10°; a scattering angle of the medium-angle-scattered light signal is 10° to 20°; a scattering angle of the high-angle-scattered light signal is 20° to 70°; a scattering angle of the side-scattered light signal is 70° to 110°; and a scattering angle of the backward-scattered light signal is 110° to 160°.

In an embodiment of the present application, any two or all of the forward-scattered signals, the medium-angle-scattered signals, and the high-angle-scattered signals are collected for subsequent classification of the red blood cells and the platelets in the blood sample, and using at least two of the above-mentioned three types of scattered light signals can achieve more effective and accurate classification of the red blood cells and the platelets. Specifically, for example, in a scattergram of red blood cells and platelets obtained by using at least two types of the forward-scattered signals, the medium-angle-scattered signals, and the high-angle-scattered signals, there is a clearer boundary between the red blood cells and the platelets, and the classification result is thus more accurate.

Certainly, it should be noted that the selection of scattered light signals is not limited to the above example, and may be done according to actual needs.

The light irradiation may be irradiation with natural light or light of a specific wavelength band, or may be polarized light irradiation in specific states of polarization. When the test sample solution is irradiated with polarized light, the scattered light signals are signals in specific states of polarization of the axial light loss, the forward-scattered light signals, the medium-angle-scattered light signals, the high-angle-scattered light signals, the side-scattered light signals, and the backward-scattered light signals that are generated by the particles in the sample solution under the polarized light irradiation.

Similarly, in an embodiment of the present application, any two or all of the signals in specific states of polarization of the forward-scattered signals, the medium-angle-scattered signals, and the high-angle-scattered signals are collected for subsequent classification of the red blood cells and the platelets in the blood sample, to obtain more effective and accurate classification of the red blood cells and the platelets.

Step S240: the red blood cells and the platelets in the test sample solution are classified according to the at least two types of scattered light signals.

For example, the processor 140 receives the scattered light signals from the optical detection apparatus 120 and processes the scattered light signals, to obtain a classification result of the red blood cells and the platelets in the blood sample to be tested.

Preferably, in step S240, the step of classifying the red blood cells and the platelets may comprise: generating, according to the at least two types of scattered light signals, a two-dimensional or three-dimensional scattergram of the particles in the blood sample; and classifying the red blood cells and the platelets in the blood sample based on the two-dimensional or three-dimensional scattergram.

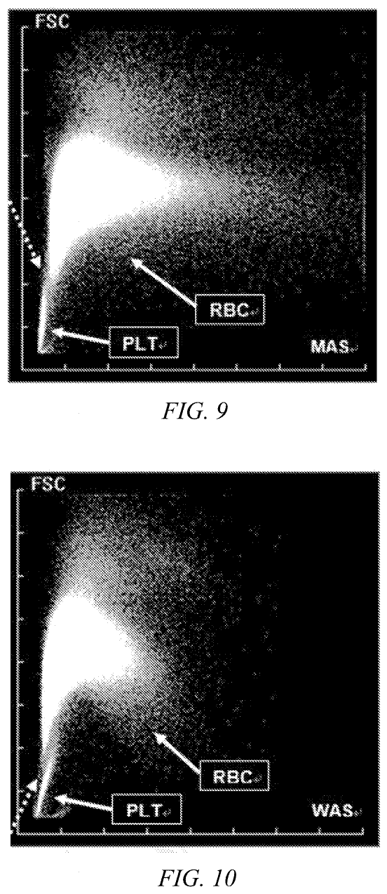

In an embodiment of the present application, for example, when a PLT count of the test sample solution is less than 30, an area to the right of a boundary of a PLT histogram is jagged, as shown in FIG. 3, and it is difficult to draw a boundary for separation from an RBC area. However, with the analyzing method described in the present application, scattergrams as shown in FIGS. 9 and 10 can be generated. FIG. 9 is a scattergram of forward-scattered (FSC) light signals versus medium-angle-scattered (MAS) light signals. FIG. 10 is a scattergram of forward-scattered (FSC) light signals versus high-angle-scattered (WAS) light signals. It can be seen from FIGS. 9 and 10 that there is a clear boundary between a PLT particle population and an RBC particle population, as shown by the dashed line in the figures. Therefore, the PLT and RBC particle populations that cannot be distinguished in the impedance method can be distinguished in the scattergram of light scattering, so that accurate RBC and PLT measurement results can be obtained.

In addition to classification of the red blood cells and the platelets, a measurement result of a predetermined parameter of the red blood cells and the platelets may also be obtained according to the at least two typed of scattered light signals. The predetermined parameter may comprise at least one parameter selected from a red blood cell count, a platelet count, a mean corpuscular volume (MCV), a mean platelet volume (MPV), and a red blood cell distribution width (RDW), and other parameters calculated from a combination of the foregoing parameters.

Further, the method 200 may further comprise outputting the classification result and/or the predetermined parameter of the red blood cells and the platelets of the blood sample to be tested.

The method 200 for measuring red blood cells and platelets in a blood sample according to the first implementation of the present application and the corresponding sample analyzer can achieve accurate classification and counting of the RBCs and the PLTs, especially achieve accurate classification and counting of the RBCs and the PLTs using a general diluent.

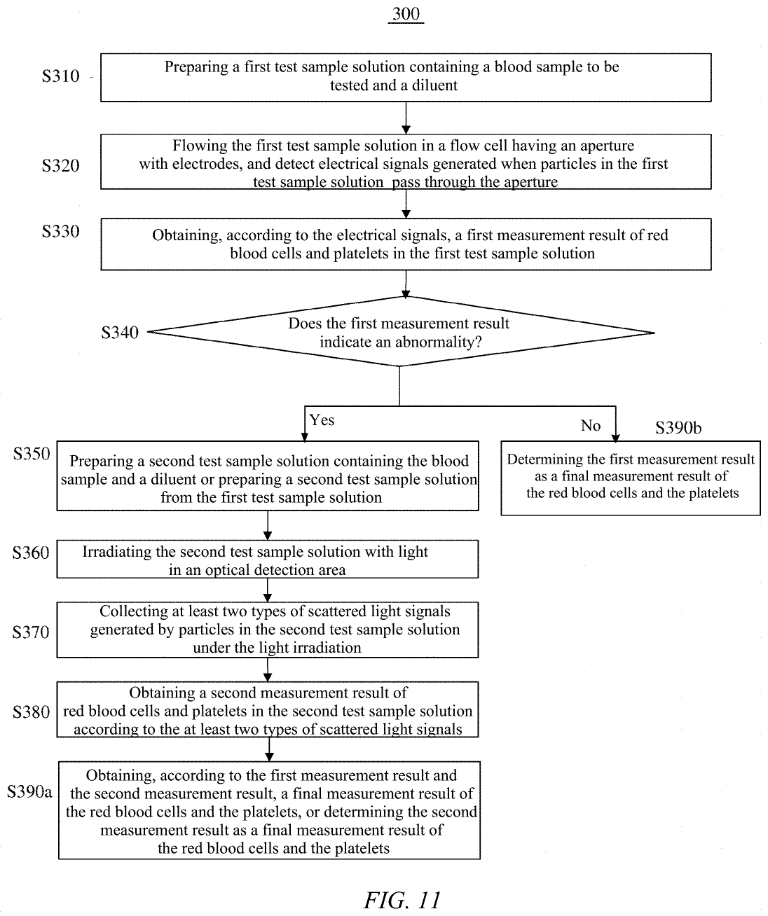

Next, a sample analyzing method for measuring red blood cells and platelets in a blood sample provided in a second implementation of the present application will be described in detail with reference to FIG. 11. As shown in FIG. 11, the method 300 comprises:

Step S310: a first test sample solution containing a blood sample to be tested and a diluent is prepared.

For step S310, reference can made to step S210 in the method 200 provided in the first implementation of the present application. Certainly, further improvements and adjustments can be made according to a method of preparing a test sample solution in the impedance method. Details are not described herein again.

Step S320: the first test sample solution is flowed in a flow cell having an aperture with electrodes, and electrical signals generated when particles in the first test sample solution pass through the aperture are detected.

For example, in this step, under the control of the processor 140, the first test sample solution is transferred by the transfer apparatus 130 to the flow cell 151 of the impedance detection apparatus 150. The sheath fluid chamber of the impedance detection apparatus 150 supplies a sheath fluid to the flow cell 151, the test sample solution is wrapped by the sheath fluid to flow, and the aperture 152 turns the flow of the first test sample solution into a tiny stream, such that the particles (visible components) contained in the first test sample solution pass through the aperture 152 one by one, thereby producing changes in impedance, that is, electrical signals by the electrodes 153 on the aperture 152. A resistance signal representing a change in impedance is amplified by the amplifier and then transferred to the processor 140.

Step S330: a first measurement result of red blood cells and platelets in the first test sample solution is obtained according to the electrical signals.

For example, in this step, the processor 140 obtains the electrical signals from the impedance detection apparatus 150. The strengths of the electrical signals correspond to the volumes (sizes) of the particles, and the processor 140 performs signal processing on the electrical signals to obtain the first measurement result of the red blood cells and the platelets in the first test sample solution. That is, the first measurement result is a measurement result of the red blood cells and the platelets obtained using the impedance method. Exemplarily, the first measurement result may comprise a result of at least one parameter selected from a red blood cell count, a platelet count, a mean corpuscular volume, a mean platelet volume, and a red blood cell distribution width, or a result of a parameter calculated from a combination thereof.

Step S340: whether the red blood cells and/or the platelets in the blood sample to be tested are abnormal is determined according to the first measurement result. After the first measurement result is obtained, step S340 is performed. Preferably, the abnormality may be that the number of platelets in the blood sample to be tested is less than a predetermined threshold. When the number of platelets in the blood sample to be tested is less than the predetermined threshold, the first measurement result will be as shown in FIG. 3, in which there is often no clear boundary between a PLT histogram and an RBC histogram. A histogram of a sample having a low PLT count is jagged, and as a result, the PLT histogram and the RBC histogram cannot be accurately separated by an algorithm, and then an accurate PLT measurement result cannot be obtained.

When the first measurement result indicates that the red blood cells and/or the platelets in the blood sample to be tested are abnormal, the following steps are performed: step S350: preparing a second test sample solution containing the blood sample to be tested and a diluent or preparing a second test sample solution from the first test sample solution, that is, the second test sample solution may be prepared from a new part the blood sample to be tested in the reaction cell 111 of the sample preparation apparatus 110, or the remaining part of the first test sample solution may be directly used as the second test sample solution; step S360: irradiating the second test sample solution with light in an optical detection area; step S370: collecting at least two types of scattered light signals generated by particles in the second test sample solution under the light irradiation; and step S380: obtaining, according to the at least two types of scattered light signals, a second measurement result of red blood cells and platelets in the second test sample solution. Exemplarily, the second measurement result may also comprise a result of at least one parameter selected from a red blood cell count, a platelet count, a mean corpuscular volume, a mean platelet volume, and a red blood cell distribution width, or a result of a parameter calculated from a combination thereof. For the specific implementation of step S360 to step S380, reference can be made to step S220 to step S240 in the method 200 provided in the first implementation of the present application, and details are not described herein again.

In addition, when the first measurement result indicates that the red blood cells and/or the platelets in the blood sample to be tested are abnormal, step S390a is performed: obtaining, according to the first measurement result and the second measurement result, a final measurement result of the red blood cells and the platelets in the blood sample to be tested, that is, the first measurement result obtained using the impedance method can be corrected by using the second measurement result obtained using the light scattering method. Combining the impedance method and the light scattering method can achieve a more accurate classification result of RBCs and PLTs. For example, an RBC and PLT scattergram is obtained using the light scattering method, and after RBC and PLT population analysis, RBC and PLT populations are accurately separated in RBC and PLT histograms of the impedance method, thereby assisting RBC and PLT impedance method to obtain more accurate measurement results. Certainly, in step S390a, the second measurement result may also be directly determined as a final measurement result of the red blood cells and the platelets in the blood sample to be tested.

When the first measurement result indicates that the red blood cells and/or the platelets in the blood sample to be tested are normal, step S390b is performed: determining the first measurement result as a final measurement result of the red blood cells and the platelets in the blood sample to be tested.

In addition, after step S390, the following step may be implemented: outputting the final measurement result of the red blood cells and the platelets of the blood sample to be tested.

It should be noted that for the steps of the method 300 according to the second implementation of the present application, reference can be made to the corresponding explanations and descriptions in the foregoing method 200 according to the first implementation of the present application, and details are not described herein again.

The method for measuring red blood cells and platelets in a blood sample according to the second implementation of the present application and the corresponding sample analyzer can achieve accurate classification and counting of RBCs and PLTs of an abnormal sample using a general diluent. In application scenarios with the absence of a plurality of diluents (especially spherical diluents), for abnormal samples, the method provided in the second implementation of the present application and the sample analyzer can improve the accuracy of the measurement using the impedance method.

In addition, the present application further proposes a sample analyzing method for measuring red blood cells and platelets in a blood sample. The method differs from the sample analyzing method provided in the second implementation of the present application in that: regardless of whether the blood sample to be tested is abnormal, both the impedance method and the light scattering method proposed in the present application are used to test the blood sample to be tested and obtain a first measurement result of the impedance method and a second measurement result of the light scattering method; and then depending on actual situations, the first measurement result and the second measurement result are used to obtain a final measurement result of the red blood cells and platelets in the blood sample to be tested.

It should be understood that the features, structures, and advantages mentioned in the specification, claims, and drawings, as long as they are meaningful within the scope of the present application, can be arbitrarily combined with each other. The features, structures, and advantages described for the method of the present application are applicable to the sample analyzer of the present application in a corresponding manner, and vice versa.

The technical terms used in the embodiments of the present application are only used to describe specific embodiments and are not intended to limit the present application. In the specification, the singular forms “a”, “the”, and “said” are used to include the plural forms at the same time, unless the context clearly indicates otherwise. Further, the terms “comprise” and “include” used in the specification refers to the presence of the described features, entities, steps, operations, elements and/or components, but does not exclude the presence or addition of one or more other features, entities, steps, operations, elements, and/or components.

Corresponding structures, materials, actions, and equivalents (if any) of all apparatuses or steps and functional elements in the appended claims are intended to include any structure, material, or action for performing the function in combination with other explicitly required elements. The description of the present application is given for the purpose of embodiment and description, but is not intended to be exhaustive or to limit the present application to the disclosed form. Many modifications and changes are apparent to those of ordinary skill in the art without departing from the scope and spirit of the present application. The embodiments described in the present application can better reveal the principles and practical applications of the present application, and enable those skilled in the art to understand the present application.

The flowchart described in the present application is only an embodiment, and there may be various modifications and changes to this illustration or the steps in the present application without departing from the spirit of the present application. For example, these steps may be performed in a different order, or some steps may be added, deleted, or modified. Those of ordinary skill in the art can understand that all or some of the processes for implementing the foregoing embodiments and equivalent changes made in accordance with the claims of the present application still fall within the scope of the present application.

1. A sample analyzing method for measuring red blood cells and platelets in a blood sample, wherein the method comprises:
preparing a first test sample solution containing a blood sample to be tested and a diluent;
flowing the first test sample solution in a flow cell having an aperture with electrodes, and detecting electrical signals generated when particles in the first test sample solution pass through the aperture;
obtaining, according to the electrical signals, a first measurement result of red blood cells and platelets in the first test sample solution;
determining, according to the first measurement result, whether the red blood cells and/or the platelets in the blood sample to be tested are abnormal;
when the first measurement result indicates that the red blood cells and/or the platelets in the blood sample to be tested are abnormal,
preparing a second test sample solution containing the blood sample to be tested and a diluent or preparing a second test sample solution from the first test sample solution;
irradiating the second test sample solution with light in an optical detection area;
collecting at least two types of scattered light signals generated by particles in the second test sample solution under light irradiation; and
obtaining, according to the at least two types of scattered light signals, a second measurement result of red blood cells and platelets in the second test sample solution.

2. The method of claim 1, wherein the first measurement result and/or the second measurement result comprise(s) a result of at least one parameter selected from a red blood cell count, a platelet count, a mean corpuscular volume, a mean platelet volume, and a red blood cell distribution width, or a result of a parameter calculated from a combination thereof.

3. The method of claim 1, wherein the abnormality is that a number of the platelets in the first test sample solution is less than a predetermined threshold.

4. The method of claim 1, wherein the diluent is capable of maintaining morphology of red blood cells and platelets.

5. The method of claim 1, wherein the at least two types of scattered light signals comprise at least two types of axial light loss, forward-scattered light signals, medium-angle-scattered light signals, high-angle-scattered light signals, side-scattered light signals, and backward-scattered light signals.

6. (canceled)

7. The method of claim 7, wherein the at least two types of scattered light signals comprise at least one type of the forward-scattered signals, the medium-angle-scattered signals, and the high-angle-scattered signals.

8. The method of claim 7, wherein the at least two types of scattered light signals comprise the forward-scattered signals and the medium-angle-scattered signals, or the forward-scattered signals and the high-angle-scattered signals.

9. The method of claim 1, wherein the light irradiation is polarized light irradiation, and the at least two types of scattered light signals comprise at least two types of signals in specific states of polarization of axial light loss, forward-scattered light signals, medium-angle-scattered light signals, high-angle-scattered light signals, side-scattered light signals, and backward-scattered light signals that are generated by the particles in the second test sample solution under the polarized light irradiation.

10. (canceled)

11. The method of claim 1, wherein the step of obtaining, according to the at least two types of scattered light signals, a second measurement result of red blood cells and platelets in the second test sample solution comprises:
generating, according to the at least two types of scattered light signals, a two-dimensional or three-dimensional scattergram of the particles in the second test sample solution; and
obtaining, based on the two-dimensional or three-dimensional scattergram, the second measurement result of the red blood cells and the platelets in the second test sample solution.

12. The method of claim 1, further comprising:
when the first measurement result indicates that the red blood cells and/or the platelets in the blood sample to be tested are abnormal, obtaining, according to the first measurement result and the second measurement result, a final measurement result of the red blood cells and the platelets in the blood sample to be tested, or determining the second measurement result as a final measurement result of the red blood cells and the platelets in the blood sample to be tested;
when the first measurement result indicates that the red blood cells and/or the platelets in the blood sample to be tested are normal, determining the first measurement result as a final measurement result of the red blood cells and the platelets in the blood sample to be tested; and
outputting the final measurement result of the red blood cells and the platelets of the blood sample to be tested.

13-14. (canceled)

15. A sample analyzer, comprising:
a sampling apparatus having a pipette with a pipette nozzle and having a driving apparatus for driving the pipette to quantitatively aspirate a blood sample through the pipette nozzle;
a sample preparation apparatus having a reaction cell and a liquid supply part, wherein the reaction cell is configured to receive the blood sample aspirated by the sampling apparatus, and the liquid supply part is configured to supply a diluent to the reaction cell, such that the blood sample aspirated by the sampling apparatus is mixed in the reaction cell with the diluent supplied by the liquid supply part, to prepare a test sample solution;
an impedance detection apparatus comprising a first flow cell having an aperture with electrodes, wherein the impedance detection apparatus is configured to detect DC impedances generated when particles in the test sample solution pass through the aperture, and output electrical signals reflecting information about the passage of the particles through the aperture;
an optical detection apparatus having a light source, a second flow cell, and optical collectors, wherein the particles in the test sample solution that has been treated with the diluent are capable of flowing in the flow cell, light emitted by the light source irradiates the particles in the second flow cell to generate at least two types of scattered light signals, and the optical collectors are configured to collect the at least two types of scattered light signals;
a transfer apparatus configured to transfer the test sample solution that has been treated with the diluent in the reaction cell to the impedance detection apparatus and the optical detection apparatus; and
a processor communicatively connected to the sampling apparatus, the sample preparation apparatus, the impedance detection apparatus, the optical detection apparatus, and the transfer apparatus and configured to:
instruct the sample preparation apparatus to prepare a first test sample solution containing a blood sample to be tested and a diluent;
instruct the transfer apparatus to transfer the prepared first test sample solution to the first flow cell;
obtain, from the impedance detection apparatus, electrical signals generated when the first test sample solution passes through the first flow cell of the impedance detection apparatus;
obtain, according to the electrical signals, a first measurement result of red blood cells and platelets in the first test sample solution;
determine, according to the first measurement result, whether the red blood cells and/or the platelets in the blood sample to be tested are abnormal;
when the first measurement result indicates that the red blood cells and/or the platelets in the blood sample to be tested are abnormal,
instruct the sample preparation apparatus to prepare a second test sample solution containing the blood sample to be tested and a diluent or prepare a second test sample solution from the first test sample solution;
instruct the transfer apparatus to transfer the prepared second test sample solution to the second flow cell;
obtain, from the optical detection apparatus, at least two types of scattered light signals generated by particles in the second test sample solution in the second flow cell of the optical detection apparatus under light irradiation, and
obtain, according to the at least two types of scattered light signals, a second measurement result of red blood cells and platelets in the second test sample solution.

16. The sample analyzer of claim 15, wherein the first measurement result and/or the second measurement result comprise or comprises a result of at least one parameter selected from a red blood cell count, a platelet count, a mean corpuscular volume, a mean platelet volume, and a red blood cell distribution width, or a result of a parameter calculated from a combination thereof.

17. The sample analyzer of claim 15, wherein the abnormality is that a number of the platelets in the first test sample solution is less than a predetermined threshold.

18. (canceled)

19. The sample analyzer of claim 15, wherein the diluent is capable of maintaining morphology of red blood cells and platelets.

20. The sample analyzer of any one of claim 15, wherein the at least two types of scattered light signals comprise at least two types of axial light loss, forward-scattered light signals, medium-angle-scattered light signals, high-angle-scattered light signals, side-scattered light signals, and backward-scattered light signals.

21. (canceled)

22. The sample analyzer of claim 20, wherein the at least two types of scattered light signals comprise at least one type of the forward-scattered signals, the medium-angle-scattered signals, and the high-angle-scattered signals.

23. The sample analyzer of claim 15, wherein the light source is configured as a light source emitting polarized light, and the at least two type of scattered light signals comprise at least two types of signals in specific states of polarization of axial light loss, forward-scattered light signals, medium-angle-scattered light signals, high-angle-scattered light signals, side-scattered light signals, and backward-scattered light signals that are generated by the particles in the second test sample solution under the polarized light irradiation.

24. The sample analyzer of claim 23, wherein the at least two types of scattered light signals comprise at least one type of signals in specific states of polarization of the forward-scattered signals, the medium-angle-scattered signals, and the high-angle-scattered signals.

25. The sample analyzer of claim 15, wherein the processor is further configured to:
generate, according to the at least two types of scattered light signals, a two-dimensional or three-dimensional scattergram of the particles in the second test sample solution; and
obtain, based on the two-dimensional or three-dimensional scattergram, the second measurement result of the red blood cells and the platelets in the second test sample solution.

26. The sample analyzer of claim 15, wherein the processor is further configured to:
when the first measurement result indicates that the red blood cells and/or the platelets in the blood sample to be tested are abnormal, obtain, according to the first measurement result and the second measurement result, a final measurement result of the red blood cells and the platelets in the blood sample to be tested, or determine the second measurement result as a final measurement result of the red blood cells and the platelets in the blood sample to be tested;
when the first measurement result indicates that the red blood cells and/or the platelets in the blood sample to be tested are normal, determine the first measurement result as a final measurement result of the red blood cells and the platelets in the blood sample to be tested; and
output the final measurement result of the red blood cells and the platelets of the blood sample to be tested.

27-28. (canceled)
